(19) United States
(12) Patent Application Publication
(10) Pub. No.: US 2014/0180089 A1
(43) Pub. Date: Jun. 26, 2014

(54) SYSTEM AND METHOD FOR GUIDEWIRE CONTROL

(71) Applicant: VOLCANO CORPORATION, San Diego, CA (US)

(72) Inventors: Howard Alpert, El Dorado Hills, CA (US); Bret Millett, Folsom, CA (US)

(73) Assignee: VOLCANO CORPORATION, San Diego, CA (US)

(21) Appl. No.: 14/134,565

(22) Filed: Dec. 19, 2013

Related U.S. Application Data
(60) Provisional application No. 61/745,328, filed on Dec. 21, 2012.

(51) Int. Cl. A61M 25/01; A61B 8/00; A61M 25/09

(52) U.S. Cl. CPC A61M 25/0158; A61M 25/0905; A61B 8/4444
USPC 600/438; 604/528; 604/95.01; 604/510

(57) ABSTRACT

The invention generally relates to guidewires for intravascular procedures that include an electroactive polymer. An electroactive polymer can be at one or a number of locations on or within a guidewire. The polymer reacts to an applied electrical potential by changing a dimension (e.g., contracting or expanding). Electroactive polymers can be disposed along or within the guidewires in any pattern, such as wrapped helically within a surface, placed longitudinally parallel to an axis of the guidewire, dispose circumferentially around the guidewire, others, or a combination thereof. Depending on the designed geometry, a potential difference applied by an actuator will cause the guidewire to change a shape, a property, a surface characteristic, a dimension, or a combination thereof.

### RELATED APPLICATION

This application claims priority to and the benefit of U.S. Provisional Ser. No. 61/745,328, filed Dec. 21, 2012, which is incorporated by reference in its entirety.

### FIELD OF THE INVENTION

The invention generally relates to guidewires for intravascular procedures that include an electroactive polymer.

### BACKGROUND

Some people are at risk of having a heart attack or stroke due to fatty plaque buildups in their arteries that restrict the flow of blood or even break off and block the flow of blood completely. Angioplasty is a procedure for treating sites that are affected by plaque. In this procedure, a needle is used to make an opening through a patient's skin. A guidewire is then inserted through the hole and guided through an artery and to the affected site. The physician tries to guide the wire by twisting and manipulating the proximal end that sits outside the patient.

The guidewire is meant to help in a number of treatment options. For example, an imaging guidewire (e.g., with an ultrasound or optical imaging sensor) can be used to visualize the affected site. Forward-looking ultrasound can be used to measure blood velocity by Doppler. If the affected blood vessel is severely narrowed by plaque buildup, the guidewire can be used in various treatment procedures. In angioplasty procedures, a balloon or stent is delivered to the affected site in hopes of opening up the narrowed vessel. If the affected site is totally blocked, the guidewire or a specialized tool can be used to cut through the blockage.

A number of problems are associated with these procedures. For example, a guidewire needs to be stiff enough to be pushed deep into vessels, but floppy enough to pass around curves. A good amount of one compromises the other. Also, in places where the guidewire lies against the side of the vessel, velocity measurements are unreliable due to the fact that fluids flow slowly adjacent to walls and the real velocity is not represented. Curved vessels also present navigational challenges. For example, where a curve in the vessel lies close to a branch-point, it can be difficult to guide the tip of the wire into the correct branch due to the strong tendency of the curve to push the wire towards one side of the vessel. Additionally, some of the intended procedures require additional tools that are bulky and do not perform well. For example, cutting through a chronic total occlusion can involve use of expensive and complicated laser or RF ablation tools.

### SUMMARY

The invention provides a guidewire for an intravascular procedure that includes an electroactive polymer at one or a number of locations on or within the guidewire. The polymer reacts to an applied electrical potential by changing a dimension (e.g., contracting or expanding). Electroactive polymers can be disposed along or within the guidewires in any pattern, such as wrapped helically within a surface, placed longitudinally parallel to an axis of the guidewire, dispose circumferentially around the guidewire, others, or a combination thereof. Depending on the designed geometry, a potential difference applied by an actuator will cause the guidewire to change a shape, a property, a surface characteristic, a dimension, or a combination thereof. For example, a tip of a guidewire can be made to turn rough or to exhibit teeth. The tip can also be made to vibrate, oscillate, or reciprocate, thus functioning as a saw to cut through an occlusion. A portion of the guidewire can be made to expand, to center the guidewire in a vessel. Portions of the guidewire can be made to selectively become stiff or floppy, for example. Further, a guidewire can be operated by a computer that stores a pattern of actuator signals in memory and can apply those signals to cause the guidewire to exhibit shapes. By these means, a guidewire of the invention can be caused to snake through a vessel having a complex pattern, center itself for an imaging operation or a velocity measurement, cut through plaque, steer through branched vessels to carry a balloon to an affected site, or perform other operations. A physician can use the guidewire to examine and treat arterial plaque, thereby intervening before the condition causes a stroke or heart attack.

In certain aspects, the invention provides a guidewire for an intravascular procedure that has an extended body with a proximal portion, a distal portion, and a distal tip; an electroactive polymer disposed within the body; and an actuator mechanism operable to carry a current from the proximal portion to the electroactive polymer. The electroactive polymer may be configured so that, when activated via the actuator mechanism, the distal portion curves and pulls the tip away from an axis of the guidewire. Additionally or alternatively, the electroactive polymer may configured so that the actuator mechanism vibrates a portion of the guidewire. The guidewire may be connected to, or controlled by, a computer system (e.g., including a tangible, non-transitory memory coupled to a processor) that is operable to receive a navigational input, introduce a curve at a tip of the guidewire according to the navigational input, and translate—as the guidewire is slid further into a vessel—the introduced curve along the guidewire away from the tip so that successive portions of the guidewire exhibit the introduced curve successively.

Through the computer, one may introduce a series of curves into the guidewire by means of signals issue from the computer system and acting via the electroactive polymer; store a description of the series of curves in the memory; and translate, while the guidewire is being moved in a direction along an axis of the guidewire, the series of curves along the guidewire in a direction opposite the direction of pushing, so that the guidewire passes through a lumen that is curved substantially similarly to the series of curves.

In some embodiments, activation of an electroactive polymer causes the distal tip to exhibit an altered shape (e.g., a saw-tooth, chisel, point, concave, hollow). Activation of the electroactive polymer may be used to cause a pullback and an extension (e.g., in a jackhammer or pile-driver modality). Activation of the electroactive polymer may exhibit torque on the guidewire, causing the distal tip to rotate relative to the proximal portion. Additionally, in some embodiments, the guidewire may include an Archimedes screw disposed at the distal portion, or a latent Archimedes screw that appears upon activation of the electroactive polymer. In certain embodiments, activation of the electroactive polymer causes the guidewire to center itself in a vessel. The guidewire can include a sensor, such as an imaging device or a velocity sensor (e.g., the sensor may be a forward-looking ultrasound transducer and the detection operation includes a velocity determination).

In related aspects, the invention provides a method of performing an intravascular procedure by inserting, into a vessel, a guidewire that has an extended body with a proximal portion, a distal portion, and a distal tip; using an actuator mechanism to create a potential difference; and changing, by means of the potential difference, a dimension of an electroactive polymer disposed within the body.

Methods may further include using a computer system comprising a tangible, non-transitory memory coupled to a processor, for receiving a navigational input, introducing a curve at a tip of the method according to the navigational input, and translating—as the guidewire is slid further into a vessel—the introduced curve along the guidewire away from the tip so that successive portions of the guidewire exhibit the introduced curve successively. In some embodiments, the methods include introducing a plurality of curves into the guidewire by means of signals issue from the computer system and acting via the electroactive polymer; storing a description of the plurality of curves in the memory; and translating, while the guidewire is being moved in a direction along an axis of the guidewire, the series of curves in a direction opposite the direction of pushing, so that the guidewire passes through a lumen that is curved substantially similarly to the plurality of curves.

### BRIEF DESCRIPTION OF THE DRAWINGS

FIG. 1 shows a catheter with a guidewire.

FIG. 2 shows a guidewire.

FIG. 3 gives a cross-sectional view through a catheter and guidewire.

FIG. 4 illustrates use of a guidewire to navigate to an affected artery.

FIG. 5 shows a distal tip of a guidewire.

FIGS. 6 and 7 depict use of an electroactive guidewire to curve a distal tip.

FIGS. 8 and 9 depict use of an electroactive guidewire to vibrate the distal tip.

FIG. 10 shows use of a system of the invention to trace a guidewire through curves.

FIG. 11 gives a detail view of a distal tip.

FIGS. 12-14 show a distal tip with an altered shape from an electroactive polymer.

FIG. 15 diagrams a reciprocating embodiment.

FIG. 16 illustrates a pile-driver embodiment.

FIG. 17 shows an Archimedes' screw.

FIG. 18 shows a centering mechanism.

FIG. 19 shows an expanded centering mechanism.

FIG. 20 shows centering a guidewire.

FIGS. 21-23 show electroactive struts for centering.

FIG. 24 is a system diagram according to certain embodiments.

### DETAILED DESCRIPTION

The present invention relates to a guidewire for a coronary procedure that includes an electroactive polymer (EAP) that can change size, bend, rotate, torque, reciprocate, change shape, scrape, pick, change stiffness, push, pull, pry, or make other motions, forces, and changes.

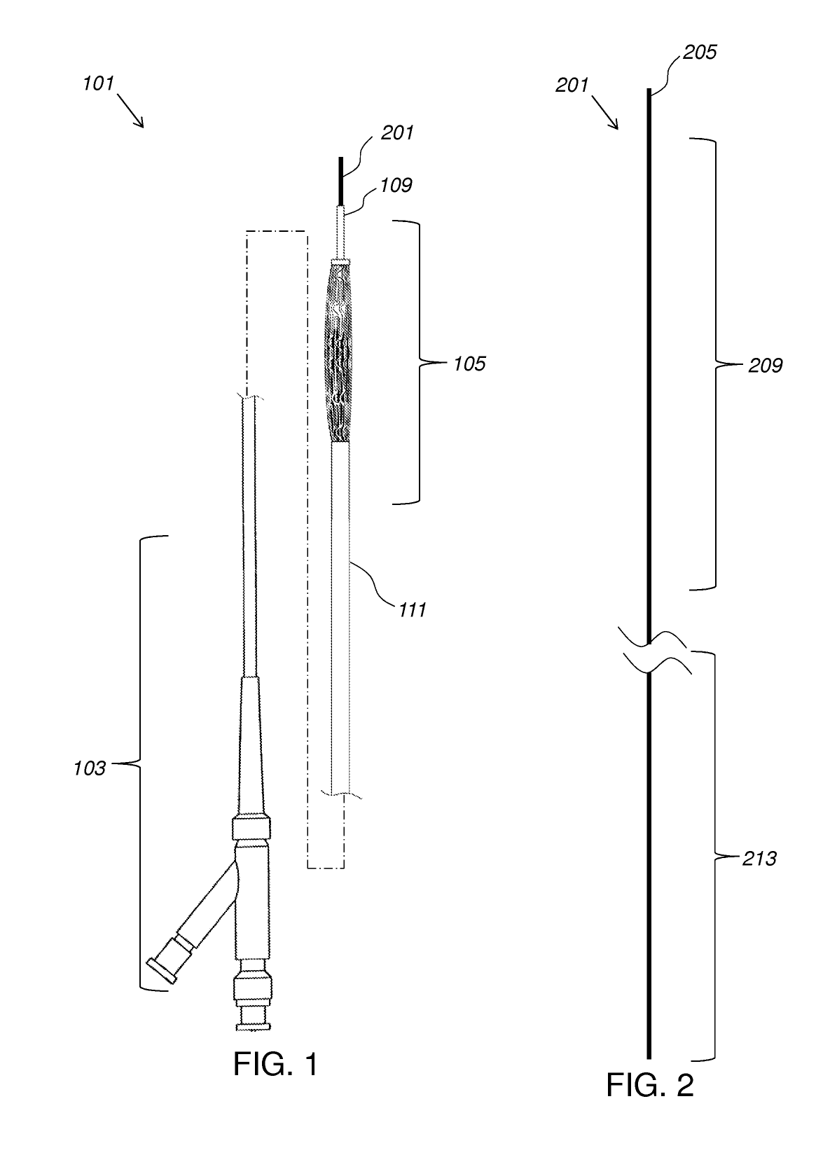

FIG. 1 shows a catheter 101 with a guidewire 201 disposed therethrough. Catheter 101 generally includes a proximal portion 103 extending to a distal portion 111. Optionally, a therapeutic device 105, such as a balloon or stent, may be located near distal tip 109.

FIG. 2 shows guidewire 201 including a proximal portion 213 extending to a distal portion 209 and terminating at distal tip 205. Guidewire 201 includes one or more electroactive polymer region and can exhibit useful properties via the electroactive polymer. Any property associated with a dimensional change in response to an applied potential may be included. Exemplary properties include variable stiffness due to the inclusion of at least one section of electroactive polymer at one or more different locations on the guidewire. In certain embodiments, actuation of the electroactive polymer causes the region surrounding the electroactive polymer section to increase in stiffness, thereby increasing the pushability of the guidewire. Alternatively, actuation of the at least one section of electroactive polymer causes the region surrounding the electroactive polymer section to decrease in stiffness, thereby increasing flexibility of the guidewire. In one embodiment, the at least one section of electroactive polymer forms part of either the inner or outer shaft of a guidewire. In one embodiment, the at least one section of electroactive polymer is a longitudinal strip. In one embodiment, the guidewire shaft is manufactured of electroactive polymer. In one embodiment, the at least one section of electroactive polymer forms the outer surface of the inner shaft. In one embodiment, the at least one section of electroactive polymer is located in the guidewire tip. In one embodiment, a braid is used as the electrode for the at least one electroactive polymer section in the variable stiffness guidewire. In one embodiment, the electroactive polymer allows for better wire movement and flexibility.

An electroactive polymer can provide an ability to curve or turn, for example, to navigate the vasculature system due to strategic positioning of at least one section of electroactive polymer at different locations on the guidewire. In one embodiment, at least one section of electroactive polymer is located only on one side of the inner shaft to control the deflection of the distal tip. In one embodiment, at least one section of electroactive polymer changes the spatial configuration of the guidewire to improve steering around corners. In one embodiment, the guidewire has at least one section of electroactive polymer. In one embodiment, the guidewire tip has at least one section of electroactive polymer. In one embodiment, the at least one section of electroactive polymer in an actuated state causes the guidewire to contract axially. Motions that can be exhibited by guidewire 201 include stretching or compression, axial rotation (e.g., torque), lateral vibration, reciprocation (e.g., sawing or toothbrush motion), or any others, or a combination thereof. In some embodiments, a guidewire is used to guide a catheter 101 to a target within a vessel.

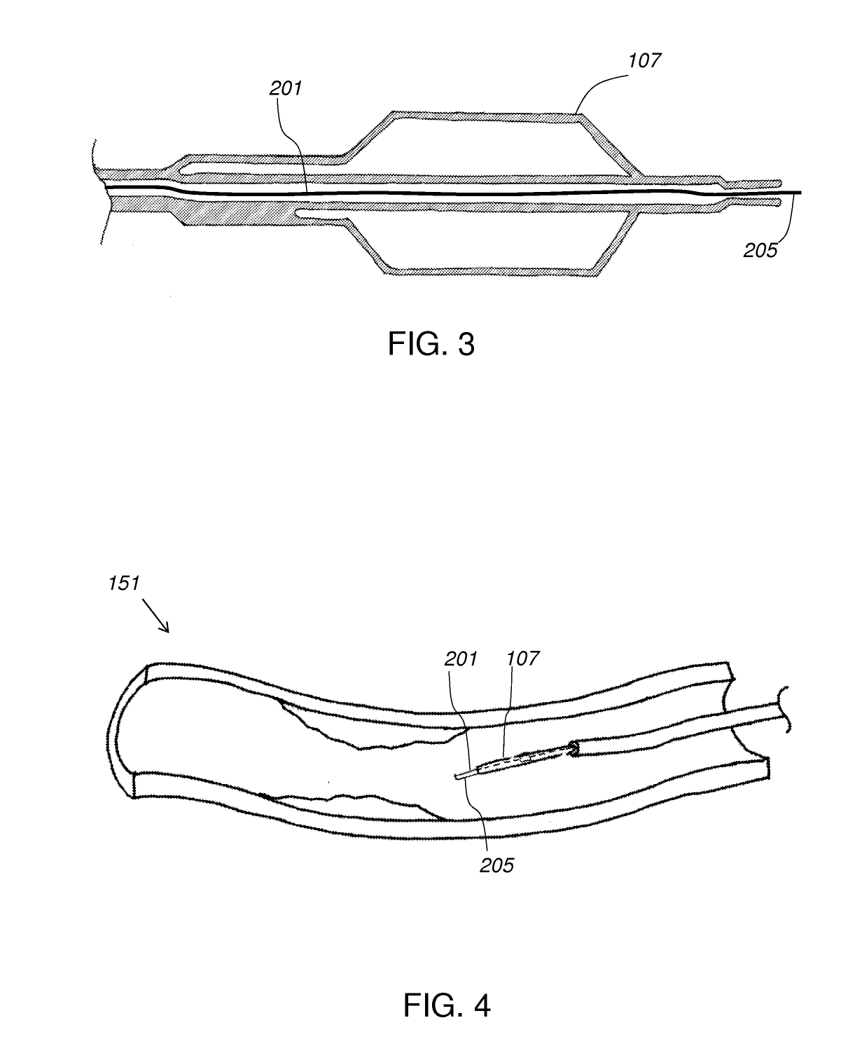

FIG. 3 gives a cross-sectional view through a catheter 101 and guidewire 201. Here, catheter 101 is depicted as including balloon 107. Distal tip 205 of guidewire 201 can be seen extending beyond the end of catheter of 101. Due to the fact that curvature of guidewire 201 can be induced from a computer workstation (e.g., by a mouse, joystick, or computer keys), guidewire 201 can be navigated through or into vessels, even where tortuous or branched. For example, a physician may refer to an angiographic display. Angiography systems can be used to visualize the blood vessels by injecting a radio-opaque contrast agent into the blood vessel and imaging using X-ray based techniques such as fluoroscopy. Angiographic techniques include projection radiography as well as imaging techniques such as CT angiography and MR angiography. In certain embodiments, angiography involves using an x-ray contrast agent and an x-ray system to visualize the arteries and guidewire 201. X-ray images of the transient radio contrast distribution within the blood flowing within the coronary arteries allows visualization of the location of guidewire 201, particularly in relation to the artery openings. A physician may refer to the angiography display to navigate guidewire 201. Angiography systems and methods are discussed, for example, in U.S. Pat. No. 7,734,009; U.S. Pat. No. 7,564,949; U.S. Pat. No. 6,520,677; U.S. Pat. No. 5,848,121; U.S. Pat. No. 5,346,689; U.S. Pat. No. 5,266,302; U.S. Pat. No. 4,432,370; and U.S. Pub. 2011/0301684, the contents of each of which are incorporated by reference in their entirety for all purposes. Useful catheters and guidewires are discussed in U.S. Pat. No. 7,766,896 and U.S. Pat. No. 7,909,844, the contents of which are incorporated by reference.

Guidewire 201 includes at least one region of electroactive polymer. Electroactive polymers deform in the presence of an applied electric field, much like piezoelectric actuators. EAPs produce force, strain, deflections, or combination thereof. In general, types of EAPs include ionic, dielectric, and composites. The ionic EAPs operate through the movement of ions within a polymer. The ionic EAPs have the potential of matching the force and energy density of biological muscles. Ionomeric polymer-metal composites (IPMC) are electroactive polymers that bend in response to an electrical activation as a result of the mobility of cations in the polymer network. Generally, two types of base polymers are employed to form IPMCs such as perfluorosulphonate sold under the trademark NAFION by Du Pont and perfluorocaboxylate sold under the trademark FLEMION by Asahi Glass, Japan. IPMC require relatively low voltages to stimulate a bending response (1-10 V) with low frequencies below 1 Hz.

Certain crystals (e.g. quartz, tourmaline and Rochelle salt), when compressed along certain axes, produced a voltage on the surface of the crystal. The reverse effect is also exhibited, whereby application of an electric current deforms the crystal. Any suitable electroactive material may be included. Suitable materials include poly(vinylidene fluoride) or PVDF and its copolymers. These materials include a partially crystalline component in an inactive amorphous phase. Applied AC fields (˜200 MV/m) induce electrostrictive (non-linear) strains of about 2%. P(VDF-TrFE) is a PVDF polymer that has been subject to electron radiation and has shown electrostrictive strain as high as 5% at lower frequency drive fields (150 V/mm).

Electrostatic fields can be employed to those polymers exhibiting low elastic stiffness and high dielectric constants to induce large actuation strain, these polymers are known as electro-statically stricted polymers (ESSP) actuators.

Ferroelectric electroactive polymer actuators can be operated in air, vacuum or water and throughout a wide temperature range.

Dielectric electroactive polymers are essentially an elastomeric capacitor. Electrostatic forces cause charged electrodes to compress an intermediate polymer layer, causing a strain response such as an expansion in a direction orthogonal to the compression. The process is also reversible, which can be used to generate electricity or be used as a sensor (much like piezoelectrics). Dielectric electroactive polymers form the basis of the electroactive polymer artificial muscle (EPAM) “spring roll” actuators. Dielectric electroactive polymer actuators can use large electric fields (˜100 V/mm) and can produce strain levels (10-200%). An acrylic elastomer tape such as the tape sold under the trademark VHB by 3M is capable of planar strains of more than 300% for biaxially symmetric constraints and linear strains up to 215% for uniaxial constraints.

Electrostrictive graft elastomers include two components, a flexible macromolecule backbone and a grafted polymer that can be produced in a crystalline form. The material exhibits high electric field induced strain (˜4%) combined with mechanical power and excellent processability. In some embodiments, the invention provides an electrostrictive-grafted elastomer with a piezoelectric poly(vinylidene fluoride-trifluoro-ethylene) copolymer. This combination has the ability to produce a varied amount of ferroelectric-electrostrictive molecular composite systems. These may be operated as a piezoelectric sensor or even an electrostrictive actuator.

Embodiments of the invention can include electro-viscoelastic elastomers that comprise a silicone elastomer and a polar phase. Upon curing, an electric field is applied that orientates the polar phase within the elastomeric matrix. An applied electric field (<6 V/mm) induces changes in shear modulus.

Liquid crystal elastomer (LCE) materials posses electroactive polymer characteristics by inducing Joule heating. LCEs are composite materials consisting of monodomain nematic liquid crystal elastomers and conductive polymers, which are distributed within their network structure. The actuation mechanism is a phase transition between nematic and isotropic phases. The actuation takes place in less than a second.

Conductive polymers (CP) includes EAPs that actuate via the reversible counter-ion insertion and expulsion that occurs during redox cycling. Significant volume changes occur through oxidation and reduction reactions at corresponding electrodes through exchanges of ions with an electrolyte. Conducive polymer actuators requires voltages in the range of 1-5 V. Variations to the voltage can control actuation speeds. Relatively high mechanical energy densities of over 20 J/cm³ are attained with these materials. Electrodes for conductive polymers may be fabricated from polypyrrole or polyaniline, or PAN doped with HCl. Other material combinations for conductive polymers are polypyrrole, polyethylenedioxythiophene, poly(p-phenylene vinylene)s, polyaniline and polythiophenes.

Carbon Nanotubes (CNT) are polymers that can be actuated via an electrolyte medium and the change in bond length via the injection of charges that affect the ionic charge balance between the nano-tube and the electrolyte. The more charges that are injected into the CNT the larger the dimension change. Due to the mechanical strength and modulus of single CNTs and the achievable actuator displacements, these electroactive polymers can boast the highest work per cycle and generate much higher mechanical stresses than other forms of electroactive polymers.

In general, the invention provides a guidewire for an intravascular procedure that includes an electroactive polymer disposed at or within a body of the guidewire. An actuator mechanism operates to carry a current from the proximal portion to the electroactive polymer. Preferably, the guidewire is an imaging guidewire, and includes an imaging sensor such as an IVUS transducer, an OCT imaging tip, or a forward-looking imaging mechanism.

The guidewire may include a size adjustment mechanism to adjust the circumferential size of the guidewire. In the embodiment, the size adjustment mechanism may operate through a pair of electroactive polymer actuators. The electroactive polymer actuators are configured to undergo deflection upon actuation to adjust the circumferential size of the guidewire.

In general, the guidewire will include one or more electroactive polymer actuator with an elastomeric polymer positioned between a pair of electrodes. The elastomeric polymer layer may be configured to deflect when a voltage difference is applied across the elastomeric polymer layer. The electroactive polymer actuator can include one or more of any of a number of polymers, including, for example, dielectric electrostrictive electroactive polymers, ion-exchange electroactive polymers, and ionomeric polymer-metal composite electroactive polymers. For certain implementations, dielectric electrostrictive electroactive polymers are particularly desirable because of their response times and operational efficiencies. Specific examples of polymers that can be used include Nusil CF19-2186 (available from Nusil Technology, Carpenteria, Calif.); dielectric elastomeric polymers; silicone rubbers; silicone elastomers; acrylic elastomers, such as VHB 4910 acrylic elastomer (available from 3M Corporation, St. Paul, Minn.); silicones, such as Dow Corning HS3 (available from Dow Corning, Wilmington, Del.); fluorosilicones, such as Dow Corning 730 (available from Dow Corning, Wilmington, Del.); acrylic polymers, such as acrylics in the 4900 VHB acrylic series (available from 3M Corporation, St. Paul, Minn.); polyurethanes; thermoplastic elastomers; copolymers including poly(vinylidene fluoride); pressure-sensitive adhesives; fluoroelastomers; polymers including silicone and acrylics, such as copolymers including silicone and acrylic and polymer blends including a silicone elastomer and an acrylic elastomer; and combinations of two or more of these polymers. Electroactive polymers are discussed in U.S. Pat. No. 8,206,429; U.S. Pat. No. 8,133,199; U.S. Pat. No. 6,514,237; U.S. Pat. No. 5,573,520; U.S. Pat. No. 4,830;023; U.S. Pub. 2012/0265268; and U.S. Pub. 2007/0208276, the contents of which are incorporated by reference.

One or more electroactive polymer may be used in a guidewire. In particular, an electroactive polymer may be used in guidewires to selectively alter the cross-section or shape of a guidewire, to alter the stiffness, to curve or bend, saw, brush, scrape, reciprocate, punch, hook, remember shapes and exhibit those shapes automatically under computer control, as well as in other ways discussed herein. Use of electroactive polymers is discussed further in U.S. Pat. No. 8,100,838; U.S. Pat. No. 8,021,377; U.S. Pat. No. 6,969,395; U.S. Pat. No. 6,139,510; U.S. Pub. 2005/0165439; and U.S. Pub. 2004/0220606, the contents of which are incorporated by reference.

FIG. 4 illustrates use of a guidewire to navigate to an affected artery. Guidewire 201 is pushed into artery 151, lead by distal tip 205 of guidewire 201. As shown here, insertion of guidewire 201 can be followed by delivery of a catheter (e.g., carrying balloon 107) for performing a treatment. Successful navigation of guidewire 201 can benefit from the one or more electroactive polymer due to the fact that an electroactive polymer can be used to stiffen guidewire 201, curve the tip, bend the guidewire, or offer other functionality. The guidewire is depicted here as carrying balloon 107. However it can be appreciated that the guidewire can be any one of multiple different intravascular or non-intravascular guidewire types. A person of ordinary skill in the art will be familiar with different types of guidewires appropriate for multiple embodiments. Some examples of other intravascular guidewires include, but are not limited to, diagnostic guidewires, atherectomy guidewires, stent delivery guidewires, and the like. In general, dilatation balloon guidewires are preferably designed to optimize pushability, trackability, crossability, and torque transmission to the distal guidewire end as such is applied to the proximal end of the guidewire. In accordance with the present invention, pushability may be defined as the ability to transmit force from the proximal end of the guidewire to the distal end of the guidewire. A guidewire shaft preferably has adequate strength for pushability and resistance to buckling or kinking. Trackability may be defined for the purpose of this application as the ability to navigate tortuous vasculature. A more flexible distal portion may improve such trackability. In accordance with the present invention, crossability may be defined as the ability to navigate the guidewire across narrow restrictions or obstructions in the vasculature.

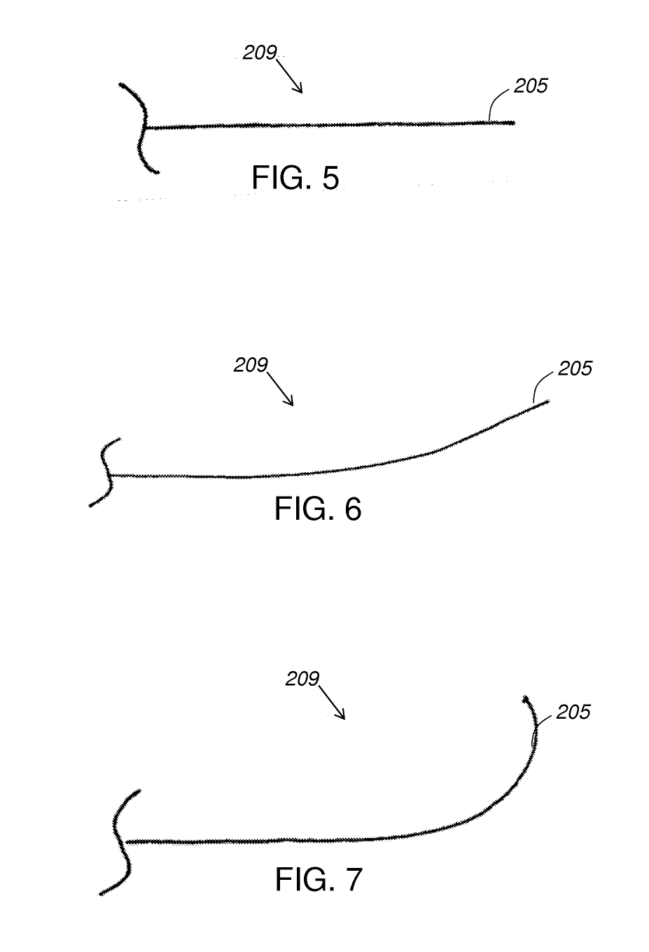

FIG. 5 shows distal tip 205 of guidewire 201. Within the material of guidewire 201 is an electroactive polymer (e.g., any of the electroactive polymers discussed herein). Application of a potential to the polymer causes the polymer to change a dimension. For example, where a potential is applied to a polymer disposed along a side of guidewire 201, and the polymer contracts, the guidewire may curve, or bend, in response.

FIGS. 6 and 7 depict use of an electroactive guidewire to curve a distal tip. Any amount of curvature may be introduced. For example, as shown in FIG. 7, a guidewire can be made to curve back on itself. Altering a shape of a guidewire to include a curve (as shown in FIGS. 6 and 7) aids in navigation through vessel 151. Additional, adding the curve includes moving the guidewire and the motion itself can provide beneficial functionality. For example, where a curving motion is repeated in opposite directions near the tip, the guidewire may wiggle or vibrate. Such a motion may be used to treat a stenosized or occluded region within a vessel.

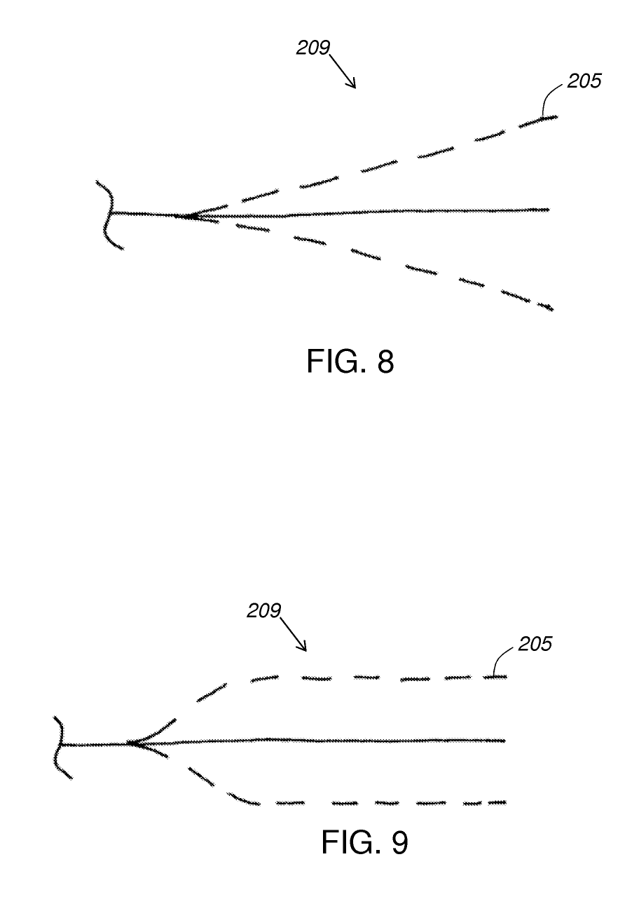

FIG. 8 shows the use of an electroactive guidewire to vibrate distal tip 205. Here, tip 205 curves first one way, then another. When repeated rapidly, this may result in a desired vibration motion. Additionally, the displacement of the tip may be controlled so that it occurs in forms other than an angular bend.

FIG. 9 depicts a non-angular vibratory motion of distal tip 205. Here, the guidewire 201 is displaced away from itself first in one direction and then in another. Each location of guidewire 201 is substantially parallel to its original location. In general, activation of an electroactive polymer may come under the control of a device such as a joystick or other mechanism and may include the use of a computer. Using a computer to activate an electroactive polymer in guidewire 201 may provide particular beneficial functionality due to the fact that a computer includes a tangible, non-transitory memory that can store information about guidewire 201. Such information can include a description of a pattern of bends (e.g., a plurality of curves) to be replicated (e.g., where the plurality of curves mimics the shape of a vessel).

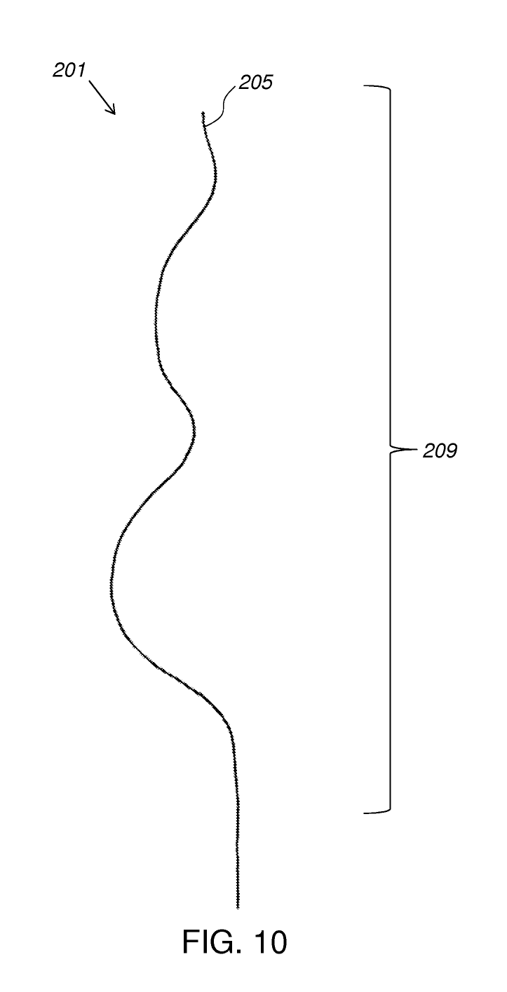

FIG. 10 shows use of a system of the invention to trace guidewire 201 through curves. Here, the invention provides systems and methods for a memory guidewire that allows a physician to slide a guidewire through its own pattern of curves and therefore through a tortuous vessel. This can begin by inserting a guidewire into a vessel (e.g., under angiographic guidance). Each curve is navigated by using a control device (e.g., joystick) to curve the tip of the guidewire so that it passes through the vessel appropriately. The attached computer stores the curve information in memory as, for example, degree of curvature and at what distance into the vessel. As subsequent portions of the guidewire pass through distance X, the computer activates the electroactive polymer at that location to curve guidewire 201 according to the stored description. Seen from a distant perspective, this can result in guidewire 201 that appears to have a plurality of curves “in memory”, as shown for example in FIG. 10. As guidewire 201 is pushed from one end, it slides through the curves, maintaining the same shape in space (e.g., within vessel 151), while the material of guidewire 201 translates along the shape in space.

Although FIGS. 5-7 show the use of electroactive polymer at distal tip 205 guidewire 201, the sections of electroactive polymer can be placed anywhere along the length of the guidewire 201 where steering control is desired. In at least one embodiment, there are a plurality of positions along the length of the guidewire 201 where there are sections of electroactive polymer about the circumference of the guidewire 201 that bend when actuated, thereby causing the guidewire 201 to bend in the region of the actuated section of electroactive polymer.

In some embodiments, at least one section of electroactive polymer forms a spiral about guidewire 201. The spiral may be, for example, a single, multiple sections of electroactive polymer or one continuous section of electroactive polymer. In at least one embodiment, there are several sections of electroactive polymer which form an overall spiral pattern. In at least one embodiment, the at least one section of electroactive polymer extends substantially the entire length of the guidewire in a spiral pattern. A spiral section of electroactive polymer can be selectively actuated to cause forced curvature or straightening of guidewire 201. For example, after a guidewire is deployed in a vessel 151 and has been used, it may lie in a curved shape which could interfere with, for example, a deployed stent while the guidewire is being withdrawn. In at least one embodiment, selective actuation will resist or prevent the inner shaft from holding, adopting, or maintaining the curvature or shape of a vessel during withdrawal of the guidewire.

As discussed herein, the actuation of the electroactive polymer improves the steering of the guidewire around corners or turns as the guidewire traverses the vasculature.

The guidewire 201 can be manufactured by co-extruding a removable nylon wire in the wall of the guidewire shaft. After the nylon wire is pulled out, the resulting shaft can be coated with a conductive ink to form the electrode and filled with an electroactive polymer by electro polymerization. The counter electrode can be a conductive ink on the outside of the guidewire shaft. Each axial section of electroactive polymer may be deposited on one fraction of the circumference of a metallic guide wire 201. A counter electrode can be deposited or printed on an insulator, which is positioned on the guide wire 201 opposite from the section of electroactive polymer. Actuation of the section of electroactive polymer causes the guidewire 201 to bend in a direction that is opposite from where the section of electroactive polymer coats the guidewire 201. Desirably, in use, these axial sections of electroactive polymer will allow the physician to control the direction of the guidewire 201 and allow for better maneuvering within the body lumen.

In at least one embodiment, the guidewire 201 includes a polymer heat shrink tube made from polyester (PET). A conductive ink, for example, but not limited to, a silver or gold ink from Erconinc can be deposited onto the PET film. Because lines of conductive ink can be made very fine, multiple conductor lines can be printed along the guidewire 201. At the position of the electroactive polymer actuator, a larger surface can be printed and the electroactive polymer deposited.

Additionally or alternatively, an electroactive polymer an be used to stiffen or un-stiffen (e.g., make floppy) select portions of guidewire 201. Guidewire 201 may include a plurality of longitudinal strips of electroactive polymer positioned about the circumference of the guidewire shaft. Multiple strips of electroactive polymer, located at the same circumferential coordinate, may be positioned along the longitudinal length of the guidewire shaft. The exact placement about the circumference of the guidewire shaft is not critical so long as the strips of electroactive polymer are located about the entire circumference of the shaft along the area(s) where control of the flexibility/rigidity of the guidewire shaft is desired. Desirably, actuation of the longitudinal strips of electroactive polymer modifies the rigidity of the guidewire shaft in the region of the electroactive polymer strips. The strips may then be used to increase the stiffness and decrease the flexibility of the guidewire. In one embodiment, the longitudinal strips decrease in size when actuated and decrease the stiffness and increase the flexibility of the guidewire. In one embodiment, longitudinal strips of electroactive polymer are positioned about the circumference of the guidewire shaft and extend from the proximal end region of the guidewire shaft to the distal end region of the guidewire shaft. In addition, the number of strips of electroactive polymer positioned about the circumference of the guidewire shaft can vary. The actuator mechanism generally includes electrodes. The electrodes of different sections of electroactive polymer are separate from one another so that precise actuation of the desired section(s) of electroactive polymer can be done. An exterior surface of a strip of electroactive polymer may be substantially flush with the exterior surface of the guidewire shaft. In some embodiments, the strip of electroactive polymer may form only a portion of the wall of the guidewire shaft, i.e. the strip of electroactive polymer does not have the same thickness as the wall of the guidewire shaft and is not flush with either the exterior surface or the interior surface of the shaft.

In certain embodiments, stiff elements, e.g. stiff polymer strips, are engaged to a layer of electroactive polymer. If a guidewire 201 with greater stiffness is desired, the layer of electroactive polymer is actuated. Actuation of the layer of electroactive polymer causes the electroactive polymer to volumetrically increase in size and moves the stiff polymer strips outwards, to cause an increase in the stiffness of the guidewire 201 because the stiffness increases with the fourth power of the size. The polymer strips may extend along the entire length of the guidewire 201 or the strips may be positioned at particular areas along the length of the guidewire 201 where control of the stiffness of the guidewire shaft is desired. Similarly, the layer of electroactive polymer may extend along the entire length of the guidewire 201 or the layer of electroactive polymer may be placed at particular areas along the length of the guidewire 201 where control of the stiffness of the guidewire shaft is desired. In one embodiment, at least one portion of the guidewire has a layer of electroactive polymer with at least one strip of stiff polymer engaged thereto. Examples of suitable materials to be used for the stiff polymer strips include, but are not limited to, polyamides, polyethylene (PE), Marlex high density polyethylene, polyetheretherketone (PEEK), polyamide (PI), and polyetherimide (PEI), liquid crystal polymers (LCP), acetal and any mixtures or combinations thereof. The polymer and actuators may be placed, for example, as described in U.S. Pub. 2005/0165439.

Additionally or alternatively, an electroactive polymer may be used to affect a surface property of guidewire 201. For example, compressing an electroactive polymer at the surface in a direction parallel to the surface can cause the electroactive polymer to expand outward, as a protrusion, lip, ridge, ring, or similar structure. Compression to cause expansion of a multiple areas near one another can create a pattern, such as a texture, or roughness, or a series of teeth, or detents, or a spiral or helical ridge. Changing a surface can be performed along the barrel of guidewire 201, over distal tip 205, or both. In certain embodiments, the shape of distal tip 205 is changed during use to enhance functionality.

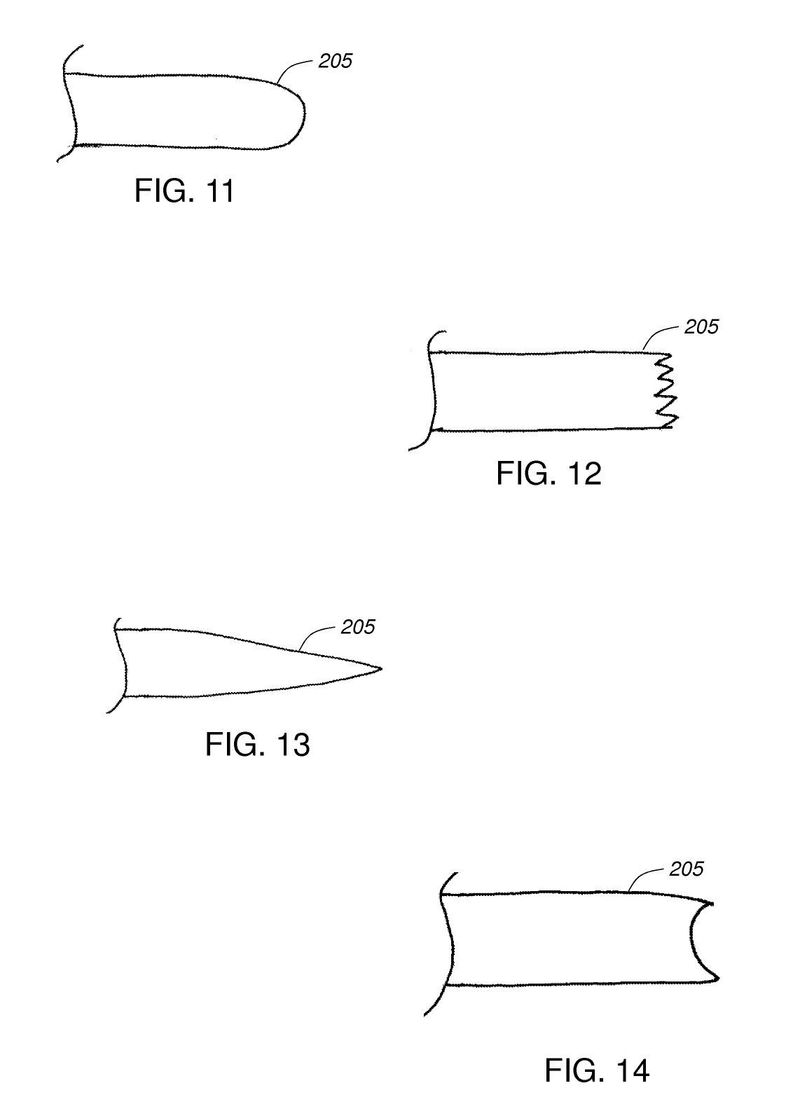

FIG. 11 gives a detail view of a distal tip 205 showing it to have a gently rounded cross section. A doctor may prefer a rounded tip for ease of insertion into vessel 151. Activation of electroactive polymer may cause tip 205 to take on a functional shape other than rounded that aids in treatment.

FIG. 12 shows a saw tooth distal tip 205. It will be appreciated that, coupled with the vibration motion as diagramed in FIG. 8 or 9, the saw tooth tip can aid in cutting through a chronic total occlusion.

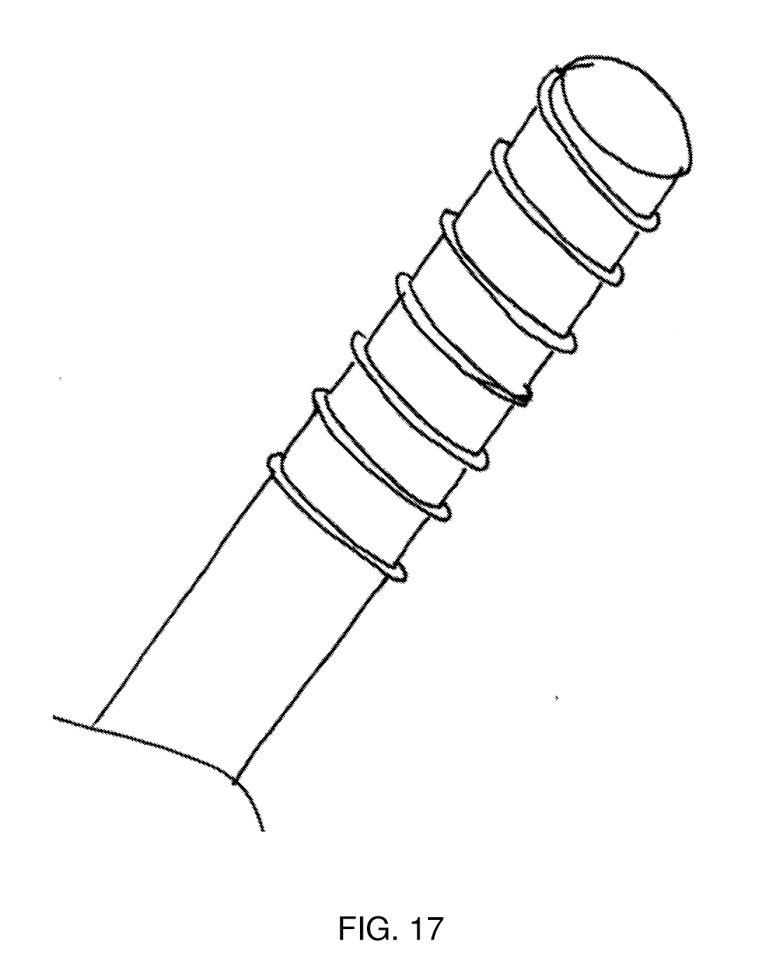

FIG. 13 shows a distal tip 205 with a point. Coupled with a pile driver motion diagrammed in FIG. 16 and discussed below, a pointed tip 205 can be useful for puncturing a chronic total occlusion.

FIG. 14 shows a concave distal tip 205 that coupled with a pile driver motion diagrammed in FIG. 16 and discussed below, could be useful for creating a punch-out hole through a chronic total occlusion.

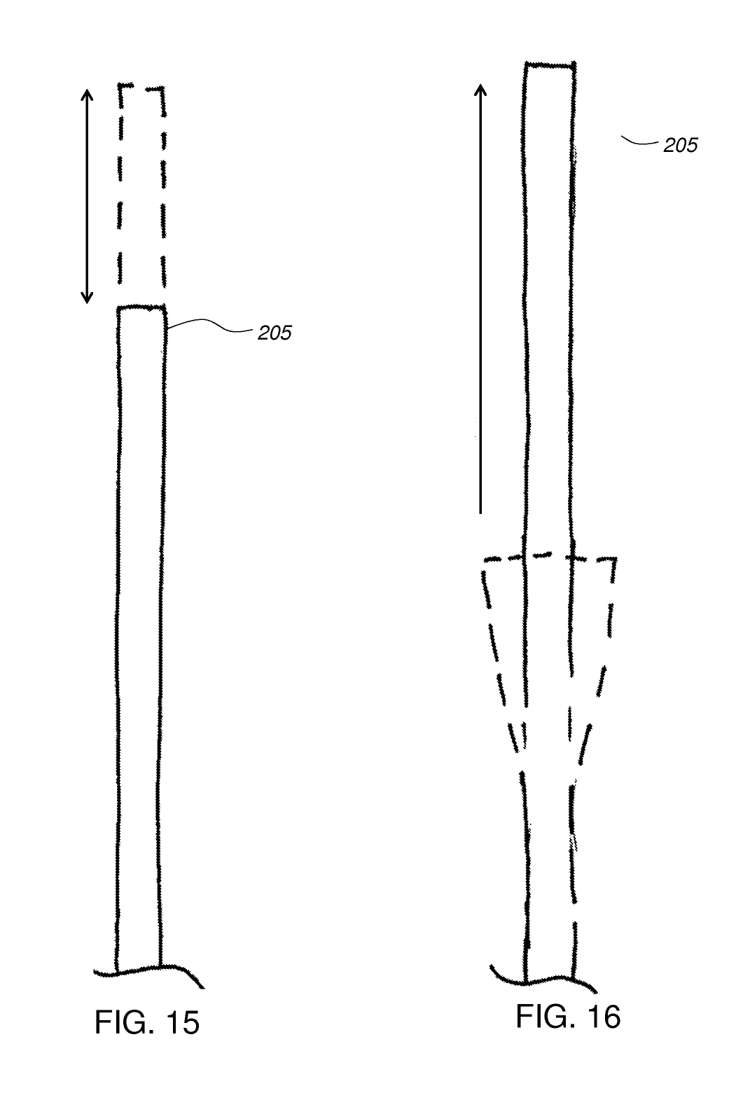

FIG. 15 diagrams a reciprocating embodiment of distal tip 205. By issuing a signal from, for example, a computer causing tip 205 to reciprocate rapidly in the pattern described by FIG. 15, guidewire 201 may be used to ablate a chronic total occlusion. Additionally or alternatively, where guidewire 201 includes, for example, a saw tooth pattern along at least one side edge, the reciprocation pattern can be used for cutting (e.g., to dislodge plaque to be caught with a venous filter).

FIG. 16 illustrates a variant embodiment of a guidewire 201 operable for a pile-driver-type action. Here, an electroactive polymer is activated to draw guidewire 201 back and then it is released. Upon release, guidewire 201 punches forth. Additionally or alternatively, guidewire 201 can be driven forth under the power of electroactive polymer. By these means, a guidewire 201 that includes an electroactive polymer can be provided that can drive through a chronic total occlusion.

In certain embodiments, a guidewire 201 that disrupts a plaque formation such as a chronic total occlusion is also configured to carry newly freed material from the site and optionally out of the body and away from sensitive internal organs.

FIG. 17 shows an Archimedes screw on a guidewire 201. Here, guidewire 201 can have a substantially smooth surface, and the Archimedes screw can be invokes via the action of an actuator and an electroactive polymer. In an alternative embodiment, guidewire 201 has a persistent Archimedes screw fashioned into the material. Using the screw, guidewire 201 can be rotated to carry material along a vessel. For example, a electroactive polymer can be activated to rotate guidewire 201. As guidewire 201 rotates, the Archimedes screw phenomenon is exhibited and material is carried (e.g., away from the heart). Where, for example, guidewire 201 is disposed substantially within a catheter 101 or other enclosure, an Archimedes screw can provide an effective way of collecting and sequestering debris from plaque.

In certain embodiments, a guidewire 201 or an associated catheter 101 includes an imaging mechanism. For example, an intravascular ultrasound (IVUS) mechanism may be included. Systems for IVUS are discussed in U.S. Pat. No. 5,771,895; U.S. Pub. 2009/0284332; U.S. Pub. 2009/0195514 A1; U.S. Pub. 2007/0232933; and U.S. Pub. 2005/0249391, the contents of each of which are hereby incorporated by reference in their entirety. In certain embodiments, an imaging mechanism is a forward-looking imaging mechanism that takes a picture from a perspective along an axis of guidewire 201. Forward looking imaging systems are discussed, for example, in U.S. Pat. No. 8,172,757; U.S. Pat. No. 7,612,773; U.S. Pub. 2012/0257210; U.S. Pub. 2011/0190586; and U.S. Pub. 2007/0287914, the contents of which are incorporated by reference. One application of forward-looking imaging is the determination of a velocity of blood within a vessel. Where, for example, a forward looking imaging system uses a sonic signal such as ultrasound, signal that is reflected and received back by the imaging sensor may have a wavelength that is shifted according to Doppler principles. Due to the velocity gradient exhibited by fluid flowing in a vessel (e.g., relatively slowly near a wall and parabolic across a section), it is desired to take a velocity reading away from a wall of a vessel. Where a guidewire lies against a wall of a vessel, the invention provides a centering mechanism that employs an electroactive polymer to push a guidewire away from a vessel wall (e.g., and bias guidewire 201 towards a center of the vessel) so that a velocity measurement may accurately reflect an amount of blood capable of flowing through the vessel.

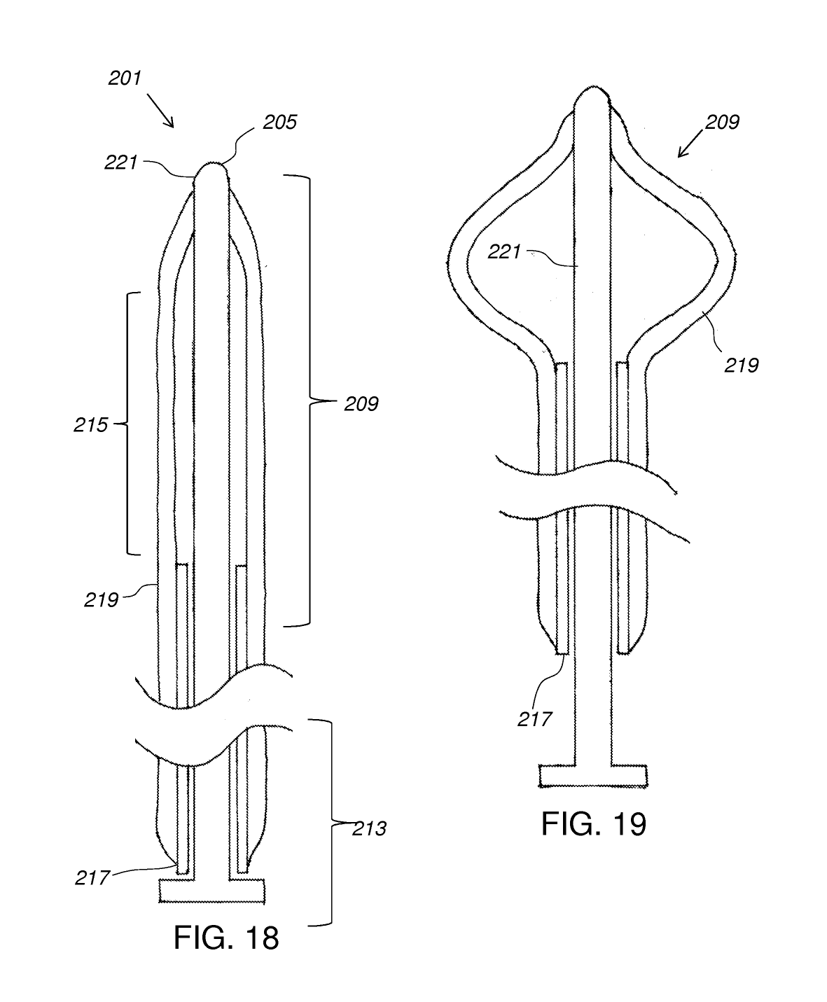

FIG. 18 shows a centering mechanism. Guidewire core 221 acts as an electrode and inner sheath 217 functions as a counter electrode. Electroactive polymer 219 extends between the electrodes. Applying a potential creates a difference between the electrodes causing a compression in unattached portion 215 of polymer 219. Since the potential difference is greater near the inside, i.e., near the electrodes, the applied stress is non-uniform, and the unattached portion 215 seeks to bow outwards.

FIG. 19 shows the unattached portion 215 bowed outwards according to the applied potential difference. The bowing outwards of the electroactive polymer 219 biases guidewire 201 away from a wall of vessel 151.

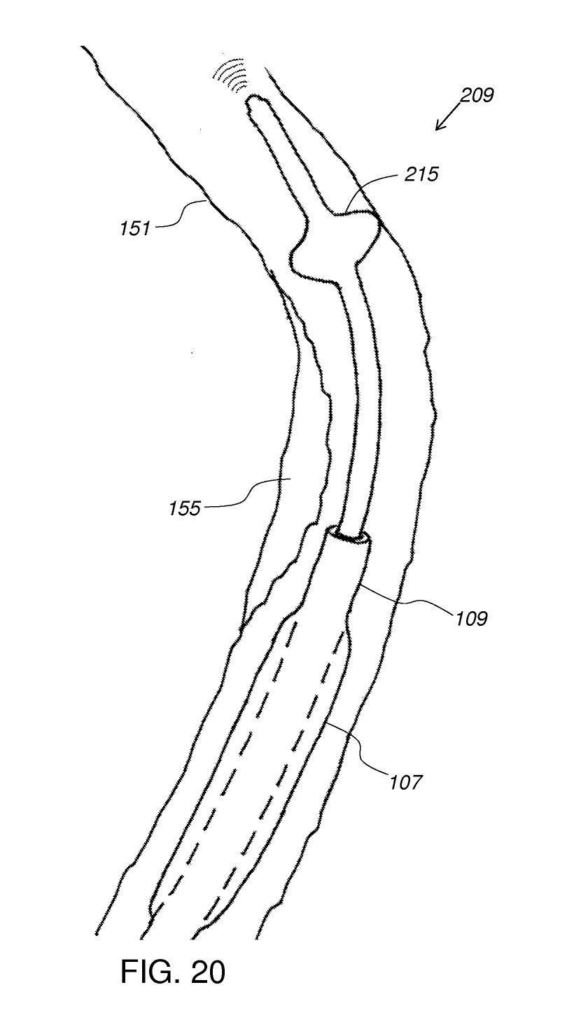

FIG. 20 shows centering guidewire based on an expansion of unattached portion 215. Here, guidewire 201 is being used for forward-looking imaging based Doppler velocity. Since guidewire 201 is not pushed up against a wall of vessel 151, the imaging operation is able to actually determine whether or not blood is flowing.

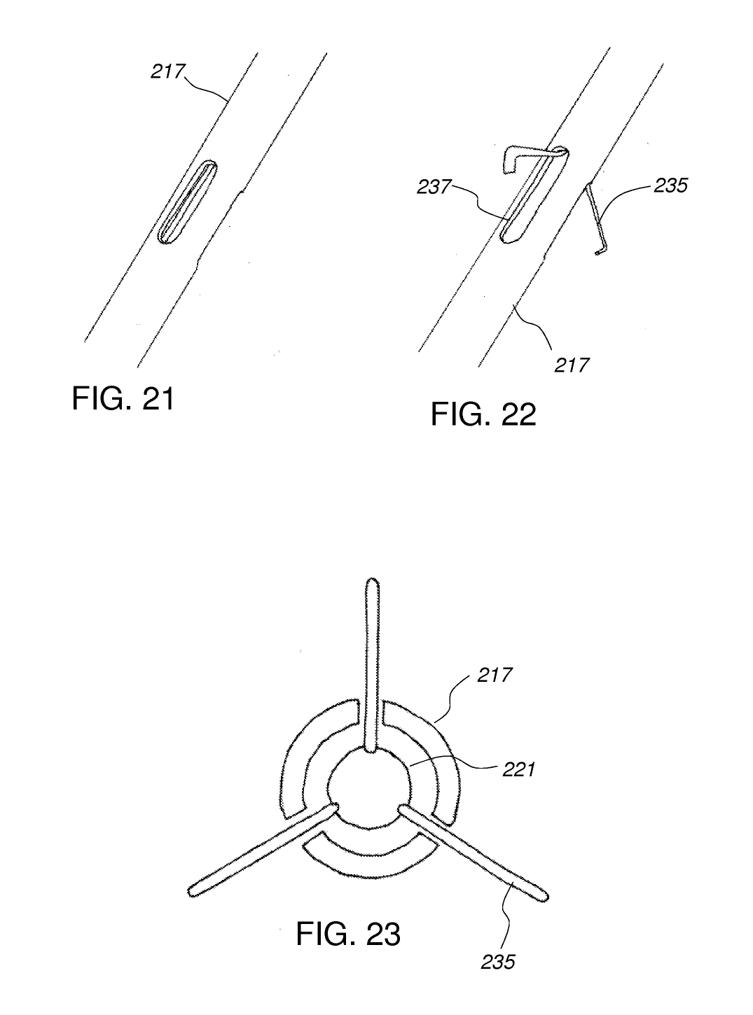

FIGS. 21-23 show electroactive struts for centering. Here, guidewire 201 is surrounded by a catheter 217, although this is a non-limiting depiction, and the centering struts 235 may be used without regard to the presence of catheter 217. Here, each strut 235 is flexibly attached at the base to guidewire shaft member 221. An electroactive polymer extends across the flexible attachment spot such that application of a potential difference causes the polymer to contract, pulling back on strut 235, causing strut 235 to expand outwardly away from guidewire body 221, thus biasing guidewire towards a center of vessel 151.

The parts of the guidewires of the present invention may be manufactured from any suitable material to impart the desired characteristics and electroactive polymers. Examples of suitable materials include, but are not limited to, polymers such as polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysulfone, nylon, perfluoro(propyl vinyl ether) (PFA), polyether-ester, polymer/metal composites, etc., or mixtures, blends or combinations thereof. One example of a polyether block ester is available under the trade name ARNITEL, and one suitable example of a polyether block amide (PEBA) is available under the trade name PEBA, from ATOMCHEM POLYMERS, Birdsboro, Pa.

The guidewires of the present invention are actuated, at least in part, using electroactive polymer actuators. Electroactive polymers are characterized by their ability to change shape in response to electrical stimulation. Electroactive polymers include electric electroactive polymers and ionic electroactive polymers. Piezoelectric materials may also be employed. Electric electroactive polymers include ferroelectric polymers, dielectric electroactive polymers, electrorestrictive polymers such as the electrorestrictive graft elastomers and electroviscoelastic elastomers, and liquid crystal elastomer materials.

Ionic EAPs include ionic polymer gels, ionomeric polymer-metal composites, conductive polymers and carbon nanotubes. Upon application of a small voltage, ionic EAPs can bend significantly. Ionic EAPs also have a number of additional properties that make them attractive for use in the devices of the present invention, including the following: (a) they are lightweight, flexible, small and easily manufactured; (b) energy sources are available which are easy to control, and energy can be easily delivered to the electroactive polymers; (c) small changes in potential (e.g., potential changes on the order of 1 V) can be used to effect volume change in the electroactive polymers; (d) they are relatively fast in actuation (e.g., full expansion/contraction in a few seconds); (e) electroactive polymer regions can be created using a variety of techniques, for example, electric deposition; and (f) electroactive polymer regions can be patterned, for example, using photolithography, if desired.

Conductive plastics may also be employed. Conductive plastics include common polymer materials which are almost exclusively thermoplastics that require the addition of conductive fillers such as powdered metals or carbon (usually carbon black or fiber).

Essentially any electroactive polymer that exhibits expansion, contraction, or other strain responses may be used in connection with the various active regions of the invention, including any of those listed above. In some embodiments, electroactive polymers include a conjugated backbone (e.g., a backbone that has an alternating series of single and double carbon-carbon bonds, and sometimes carbon-nitrogen bonds, i.e. n-conjugation) and have the ability to increase the electrical conductivity under oxidation or reduction.

The volume of these polymers changes through redox reactions at corresponding electrodes through exchanges of ions with an electrolyte. The electroactive polymer-containing active region contracts or expands in response to the flow of ions out of, or into, the same. These exchanges occur with small applied voltages and voltage variation can be used to control actuation speeds.

Any of a variety of pi-conjugated polymers may be employed herein. Examples of suitable conductive polymers include, but are not limited to, polypyrroles, polyanilines, polythiophenes, polyethylenedioxythiophenes, poly(p-phenylenes), poly(p-phenylene vinylene)s, polysulfones, polypyridines, polyquinoxalines, polyanthraquinones, poly(N-vinylcarbazole)s and polyacetylenes, with the most common being polythiophenes, polyanilines, and polypyrroles.

The behavior of conjugated polymers is dramatically altered with the addition of charge transfer agents (dopants). These materials can be oxidized to a p-type doped material by doping with an anionic dopant species or reducible to a n-type doped material by doping with a cationic dopant species. Dopants have an effect on this oxidation-reduction scenario and convert semi-conducting polymers to conducting versions close to metallic conductivity in many instances. Such oxidation and reduction are believed to lead to a charge imbalance that, in turn, results in a flow of ions into or out of the material. These ions typically enter/exit the material from/into an ionic conductive electrolyte medium associated with the electroactive polymer.

Dimensional or volumetric changes can be effectuated in certain polymers by the mass transfer of ions into or out of the polymer. This ion transfer is used to build conductive polymer actuators (volume change). For example, in some conductive polymers, expansion is believed to be due to ion insertion between chains, whereas in others inter-chain repulsion is believed to be the dominant effect. Regardless of the mechanism, the mass transfer of ions into and out of the material leads to an expansion or contraction of the polymer, delivering significant stresses (e.g., on the order of 1 MPa) and strains (e.g., on the order of 10%).

In general, use of an electroactive polymer include application of a potential to the material via electrodes. The source of the potential may include one or more of a battery, an outlet, a switch, or similar. Alternatively, more complex systems can be utilized. In at least one embodiment, for example, an electrical link can be established with a microprocessor, allowing a complex set of control signals to be sent to the electroactive polymer-containing active region(s). Other embodiments of the invention however may utilize any of a variety of electrical sources and configurations for regulating the electric current to the electroactive polymer.

An electrolyte may be in contact with at least a portion of the surface of the active region to allow for the flow of ions and thus acts as a source/sink for the ions. Any suitable electrolyte may be employed herein. The electrolyte may be, for example, a liquid, a gel, or a solid, so long as ion movement is permitted. Examples of suitable liquid electrolytes include, but are not limited to, an aqueous solution containing a salt, for example, a NaCl solution, a KCl solution, a sodium dodecylbenzene sulfonate solution, a phosphate buffered solution, physiological fluid, etc. Examples of suitable gel electrolytes include, but are not limited to, a salt-containing agar gel or polymethylmethacrylate (PMMA) gel. Solid electrolytes include ionic polymers different from the electroactive polymer and salt films.

The counter electrode may be formed from any suitable electrical conductor, for example, a conducting polymer, a conducting gel, or a metal, such as stainless steel, gold or platinum. At least a portion of the surface of the counter electrode is generally in contact with the electrolyte, in order to provide a return path for charge.

In one specific embodiment, the electroactive polymer includes polypyrrole. Polypyrrole-containing active regions can be fabricated using a number of known techniques, for example, extrusion, casting, dip coating, spin coating, or electro-polymerization/deposition techniques. Such active regions can also be patterned, for example, using lithographic techniques, if desired.

Various dopants, including large immobile anions and large immobile cations, can be used in the polypyrrole-containing active regions. According to one specific embodiment, the active region comprises polypyrrole (PPy) doped with dodecylbenzene sulfonate (DBS) anions. When placed in contact with an electrolyte containing small mobile cations, for example, sodium ions, and when a current is passed between the polypyrrole-containing active region and a counter electrode, the cations are inserted/removed upon reduction/oxidation of the polymer, leading to expansion/contraction of the same.

Electroactive polymer-containing active regions can be provided that either expand or contract when an applied voltage of appropriate value is interrupted depending, for example, upon the selection of the electroactive polymer, dopant, and electrolyte. Additional information regarding electroactive polymer actuators, their design considerations, and the materials and components that may be employed therein, can be found, for example, in U.S. Pat. No. 7,777399; U.S. Pat. No. 6,258,052; U.S. Pat. No. 6,249,076; U.S. Pat. No. 6,139,510; U.S. Pat. No. 5,693,015; U.S. Pat. No. 5,120,308; U.S. Pub. 2006/0100694; and U.S. Pub. 2006/0074442 each of which is hereby incorporated by reference in its entirety. Furthermore, networks of conductive polymers may also be employed. For example, it has been known to polymerize pyrrole in electroactive polymer networks such as poly(vinylchloride), poly(vinyl alcohol), a perfluorinated polymer that contains small proportions of sulfonic or carboxylic ionic functional groups, available from E.I. DuPont Co., Inc. (Wilmington, Del.). Electroactive polymers are also discussed in U.S. Pub. 2004/0143160 and U.S. Pub. 2004/0068161, the contents of each of which are incorporated by reference.

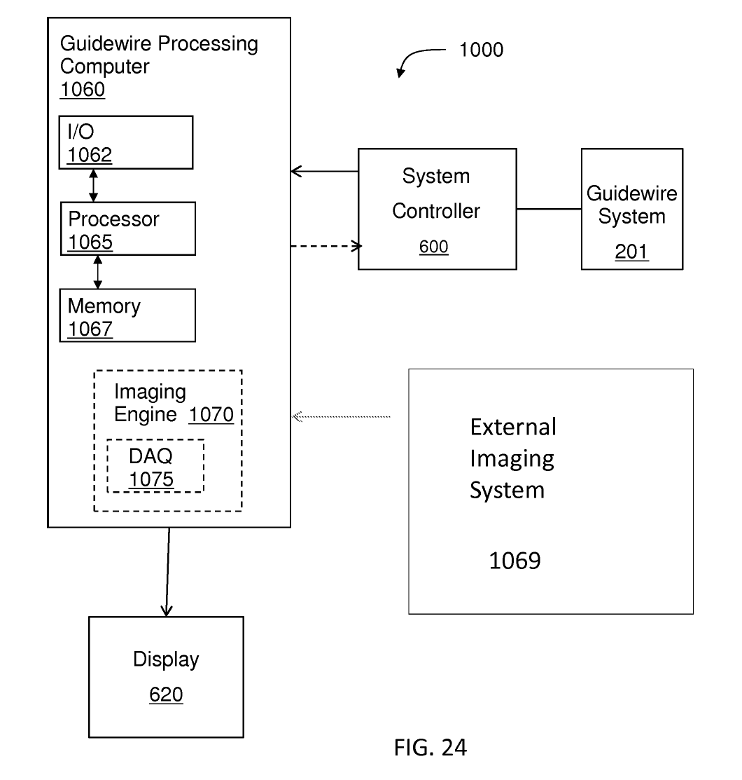

Referring now to FIG. 24, the guidewire 201 can be connected to an instrument, such as a computing 1060 (e.g. a laptop, desktop, or tablet computer) that can transmit signals to the guidewire in order to activate the electroactive polymers. The computer 1060 includes a memory 1067, a processor 1065, and I/O 1062. Alternatively, a system controller 600 may be an intermediary between the guidewire 201, and the computer system allows a user to input instructions to the system controller 600 and the system controller 600 transmits signals accordingly to activate the electroactive polymers of the guidewire. The system controller 600, the computing system 1060, or both may control the timing, duration, and amount of signal applied to activate the electroactive polymers. The system 1000 also includes a display 620 and a user interface that allow a user, e.g. a surgeon, to interact with the guidewire and to control the manipulation of the guidewire for certain applications.

The guidewire processing computer 1060 may be coupled to an external imaging system 1069 (such as an angiography system or fluoroscopy system) that transmits image data of the guidewire while disposed within body lumen. The data acquisition element 855 (DAQ) of the imaging engine 1070 receives image data from the external imaging system 1069. In some embodiments, an operator uses computer 1060 to control system 1069 or to receive images. Using the obtained image data, a user can view the guidewire 201 as disposed within the body to obtain a navigational input based on the current location of the guidewire in the body. Based on the navigational input, the computer 1060 or system controller 600 can be used to transmit signals to the guidewire 201 in order to activate the electroactive polymers based on its current location within the body and its therapeutic need at that location.

In addition to or alternatively, the guidewire processing computer 1060 is able to receive a navigational input based on imaging data obtained from one or more imaging sensors of the guidewire. In the same manner as the external imaging data, the data acquisition element 855 (DAQ) of the imaging engine 1070 receives image data from the guidewire imaging sensors. In some embodiments, an operator uses computer 1060 to control system 1069 or to receive images. Using the obtained image data, a user can view the guidewire 201 as disposed within the body to obtain a navigational input based on the current location of the guidewire in the body. Based on the navigational input, the computer 1060 or system controller 600 can be used to transmit signals to the guidewire 201 in order to activate the electroactive polymers based on its current location within the body and its therapeutic need at that location.

The computer system 1060 may be configured to receive a navigational input (e.g., provided by the user, from the external imaging data, or from the intraluminal image date), introduce a curve at a tip of the guidewire according to the navigational input, and translate, as the guidewire is slid further into a vessel, the introduced curve along the guidewire away from the tip so that successive portions of the guidewire exhibit the introduced curve successively. In certain embodiments, the computer system 1060 is further configured to introduce a plurality of curves into the guidewire by means of signals issue from the computer system and acting via the electroactive polymer, store a description of the plurality of curves in the memory, translate, while the guidewire is being moved in a direction along an axis of the guidewire, the plurality of curves along the guidewire in a direction opposite the direction of pushing, so that the guidewire passes through a lumen that is curved substantially similarly to the plurality of curves.

As used herein, the word “or” means “and or or”, sometimes seen or referred to as “and/or”, unless indicated otherwise.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A guidewire for an intravascular procedure, the guidewire comprising:
an extended body comprising a proximal portion, a distal portion, and a distal tip;
an electroactive polymer disposed within the body; and
an actuator mechanism operable to apply a potential difference to the electroactive polymer.

2. The guidewire of claim 1, wherein the electroactive polymer is configured so that, when activated via the actuator mechanism, the distal portion curves and pulls the tip away from an axis of the guidewire.

3. The guidewire of claim 1, wherein the electroactive polymer is configured so that the actuator mechanism vibrates a portion of the guidewire.

4. The guidewire of claim 1, further comprising a computer system comprising a tangible, non-transitory memory coupled to a processor, wherein the computer system is operable to:
receive a navigational input;
introduce a curve at a tip of the guidewire according to the navigational input; and
translate, as the guidewire is slid further into a vessel, the introduced curve along the guidewire away from the tip so that successive portions of the guidewire exhibit the introduced curve successively.

5. The guidewire of claim 4, wherein the computer system is further operable to:
introduce a plurality of curves into the guidewire by means of signals issue from the computer system and acting via the electroactive polymer;
store a description of the plurality of curves in the memory;
translate, while the guidewire is being moved in a direction along an axis of the guidewire, the plurality of curves along the guidewire in a direction opposite the direction of pushing, so that the guidewire passes through a lumen that is curved substantially similarly to the plurality of curves.

6. The guidewire of claim 1, wherein the distal tip is curved and further wherein activation of the electroactive polymer causes the distal tip to exhibit an altered shape.

7. The guidewire of claim 6, wherein the altered shape is one selected from the list of saw-tooth, chisel, point, concave, hollow.

8. The guidewire of claim 1, wherein activation of the electroactive polymer causes a pullback and an extension.

9. The guidewire of claim 1, wherein activation of the electroactive polymer exhibits torque on the guidewire, causing the distal tip to rotate relative to the proximal portion.

10. The guidewire of claim 9, further comprising an Archimedes screw disposed at the distal portion.

11. The guidewire of claim 1, wherein carrying current to the electroactive polymer causes the guidewire to center itself in a vessel.

12. The guidewire of claim 1, further comprising a sensor disposed on the guidewire to perform an intravascular detection operation.

13. The guidewire of claim 12, wherein the sensor comprises a forward-looking ultrasound transducer and the detection operation comprises a velocity determination.

14. A method of performing an intravascular procedure, the method comprising:
inserting into a vessel a guidewire comprising an extended body with a proximal portion, a distal portion, and a distal tip;
using an actuator mechanism to create a potential difference; and
changing, by means of the potential difference, a dimension of an electroactive polymer disposed within the body.

15. The method of claim 14, wherein the electroactive polymer is configured so that, when activated via the actuator mechanism, the distal portion curves and pulls the tip away from an axis of the guidewire.

16. The method of claim 14, wherein the electroactive polymer is configured so that the actuator mechanism vibrates a portion of the method.

17. The method of claim 14, further comprising using a computer system comprising a tangible, non-transitory memory coupled to a processor, for:
receiving a navigational input;
introducing a curve at a tip of the method according to the navigational input; and
translating, as the guidewire is slid further into a vessel, the introduced curve along the method away from the tip so that successive portions of the guidewire exhibit the introduced curve successively.

18. The method of claim 17, further comprising:
introducing a plurality of curves into the method by means of signals issue from the computer system and acting via the electroactive polymer;
storing a description of the plurality of curves in the memory;
translating, while the guidewire is being moved in a direction along an axis of the method, the series of curves in a direction opposite the direction of pushing, so that the guidewire passes through a lumen that is curved substantially similarly to the plurality of curves.

19. The method of claim 14, wherein activation of the electroactive polymer causes the distal tip to exhibit an altered shape.

20. The method of claim 19, wherein the altered shape is one selected from the list of saw-tooth, chisel, point, concave, hollow.

21. The method of claim 14, wherein activation of the electroactive polymer causes a pullback and an extension.

22. The method of claim 14, wherein activation of the electroactive polymer exhibits torque on the guidewire, causing the distal tip to rotate relative to the proximal portion.

23. The method of claim 22, wherein the guidewire comprises an Archimedes screw disposed at the distal portion.

24. The method of claim 14, wherein the electroactive polymer centers the guidewire in a vessel.

25. The method of claim 14, wherein the guidewire further comprises a sensor to perform an intravascular detection operation.

26. The method of claim 25, wherein the sensor comprises a forward-looking ultrasound transducer and the detection operation comprises a velocity determination.
